(19) United States
(12) Patent Application Publication
(10) Pub. No.: US 2013/0110257 A1
(43) Pub. Date: May 2, 2013

(54) Bleeder Valve For A Prosthetic Limb Assembly

(71) Applicant: Prosthetic Design, Inc., Clayton, OH (US)

(72) Inventors: Tracy C. Slemker, Clayton, OH (US); Robert Hoskins, Springboro, OH (US); Bradley Joseph Poziembo, Kettering, OH (US)

(73) Assignee: Prosthetic Design, Inc., Clayton, OH (US)

(21) Appl. No.: 13/662,930

(22) Filed: Oct. 29, 2012

Related U.S. Application Data
(60) Provisional application No. 61/552,976, filed on Oct. 28, 2011.

(51) Int. Cl. A61F 2/80; F16K 27/00

(52) U.S. Cl. 623/34; 251/366; 137/14

(57) ABSTRACT

A prosthetic limb socket assembly includes a bleeder valve configured to automatically adjust vacuum pressure within the prosthetic limb socket based upon a difference in ambient air pressure and vacuum pressure within the prosthetic limb socket. The bleeder valve includes: a housing having a first end, a second end, and channel extending therethrough providing fluid communication between the ambient and the interior of the prosthetic limb socket; and a valve disc disposed within the channel and biased into a substantially sealed engagement with a valve seat, wherein a certain pressure difference between the ambient and the interior of the prosthetic limb socket is operative to overcome the bias and unseat the valve disc from the valve seat.

### CROSS REFERENCE TO RELATED APPLICATIONS

The current disclosure claims the benefit of U.S. Provisional Application, Ser. No. 61/552,976, filed Oct. 28, 2011, the disclosure of which is incorporated herein by reference.

### BACKGROUND

The present disclosure generally pertains to systems for releasably coupling a prosthetic device, such as a prosthetic limb socket assembly, to a residual limb of an amputee.

The present disclosure contemplates that in some circumstances it may be desirable to apply a vacuum to a socket of a prosthetic limb to assist maintaining a patient's residual limb within the socket. Among other potential benefits, applying a vacuum to a socket may assist in retaining the socket on a wearer's residual limb and/or may reduce volume loss in the residual limb. As used herein, “vacuum” may refer to pressures less than atmospheric pressure. A vacuum may be applied to a socket by withdrawing some of the air within the socket using a vacuum pump, for example.

### SUMMARY

Embodiments of the current disclosure include a bleeder valve assembly that may be incorporated into an elevated vacuum locking system of a prosthetic limb assembly. Use of such a bleeder valve system may improve retention and comfort for a prosthetic limb assembly that includes a stump-receiving socket.

According to an embodiment, a prosthetic limb socket assembly includes a valve configured to automatically adjust vacuum pressure within the prosthetic limb socket based upon a difference in ambient air pressure and vacuum pressure within the prosthetic limb socket. In a more detailed embodiment, the valve comprises a bleeder valve. In a further detailed embodiment, the bleeder valve includes: a housing having a first end, a second end, and channel extending therethrough providing fluid communication between the ambient and the interior of the prosthetic limb socket; a valve disc disposed within the channel and biased into a substantially sealed engagement with a valve seat, where a certain pressure difference between the ambient and the interior of the prosthetic limb socket is operative to overcome the bias and unseat the valve disc from the valve seat. In a further detailed embodiment, the bias is manually adjustable. Alternatively, or in addition, the bias is provided by a spring positioned within the channel between the valve disc and a spring seat, where a spring seat may be threadedly received within the channel so that rotating of the spring seat moves the spring seat towards or away from the valve disc, depending upon direction of rotation of the spring seat, thereby respectively compressing or decompressing the spring.

According to an embodiment, a bleeder valve assembly for use with a prosthetic limb socket assembly, includes: a housing having a first end, a second end, and an interior channel, the housing being adapted to be mounted to a prosthetic limb socket assembly such that the interior channel provides fluid communication between the ambient and an interior of a prosthetic limb socket; a spring seat positioned approximate the first end of the interior channel; a valve seat positioned approximate the second end of the interior channel; a valve disc positioned adjacent the valve seat; and a spring positioned between the valve disc and the spring seat, biasing the valve disc into sealed contact with the valve seat, thereby sealing the interior channel; where a certain pressure difference between the ambient and the interior of the prosthetic limb socket is operative to overcome the bias of the spring and unseat the valve disc from the valve seat. In a more detailed embodiment, the spring seat is axially adjustable to allow for adjustment of the bias applied by the spring. In a further detailed embodiment, the spring seat is threadedly mounted with respect to the interior channel. The housing may be barrel shaped. The bleeder valve may include a first fitting disposed on the first end of the housing, where the first fitting includes a nipple and a seal between the first fitting and the housing. And the bleeder valve may further include a second fitting disposed on the second end of the housing and a seal disposed between the second fitting and the housing. The valve disc may include a resilient stopper; and the valve disc may include a conical portion coaxial with the axis of the interior channel and tapering inwardly towards the first end.

An embodiment is directed to a method for regulating vacuum pressure within a prosthetic limb socket assembly including a step of automatically adjusting vacuum pressure within the prosthetic limb socket based upon a difference in ambient air pressure and vacuum pressure within the prosthetic limb socket. In a more detailed embodiment the adjusting step is performed by a valve assembly mounted to the prosthetic limb socket, where the valve assembly provides fluid communication between the prosthetic limb socket interior and the ambient when in an unsealed orientation. In a further detailed embodiment, the valve assembly is a bleeder valve assembly. In a further detailed embodiment, the bleeder valve includes: a housing having a first end, a second end, and channel extending therethrough providing fluid communication between the ambient and the prosthetic limb socket interior; and a valve disc disposed within the channel and biased into a substantially sealed engagement with a valve seat, where a certain pressure difference between the ambient and the interior of the prosthetic limb socket is operative to overcome the bias and unseat the valve disc from the valve seat. In a further detailed embodiment, the bias is manually adjustable, and the method may further include a step of manually adjusting the bias.

### BRIEF DESCRIPTION OF THE DRAWINGS

FIG. 1 is an exploded perspective view of an example bleeder valve assembly 100 according to at least certain aspects of the present disclosure;

FIG. 2 is a partial cutaway perspective view of an example bleeder valve assembly 100 according to at least some aspects of the present disclosure;

FIG. 3 is an elevational side view of an exemplary front fitting and o-ring seal according to an embodiment; and

FIG. 4 is an elevational side view of an exemplary back fitting and o-ring seal according to an embodiment.

### DETAILED DESCRIPTION

Some example bleeder valve assemblies according to the present disclosure may be configured to be fluidicly coupled to an interior volume of a socket comprising an elevated vacuum locking system. For example, some example bleeder valve assemblies may be arranged to limit the vacuum that may be applied to a socket, such as by allowing air to enter the socket when the difference in pressure between the socket and an ambient atmosphere exceeds a predetermined set point.

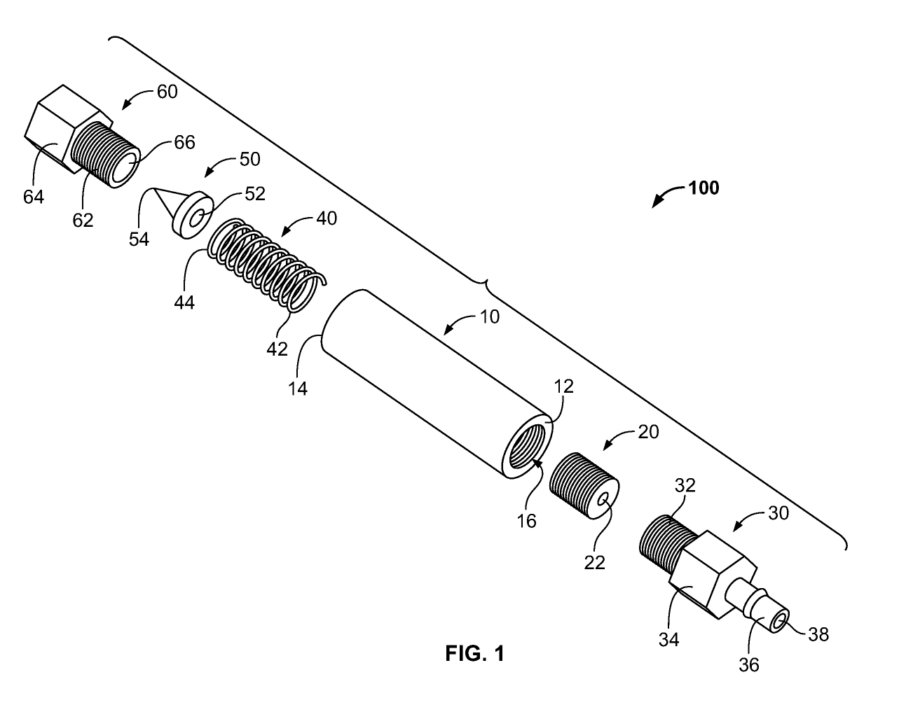

FIG. 1 is an exploded perspective view of an example bleeder valve assembly 100 according to at least certain aspects of the present disclosure. Bleeder valve assembly 100 may include a housing comprised of a generally tubular barrel 10, which may include a first end 12 and a second end 14. In some example embodiments, first end 12 and/or second end 14 may be internally threaded. Barrel 10 may at least partially define an interior 16.

A generally cylindrical, externally threaded set screw 20 may be installed into interior 16 of barrel 10, such as threading via first end 12. Set screw 20 may include an axial through passage 22, which may allow air flow longitudinally through set screw 20.

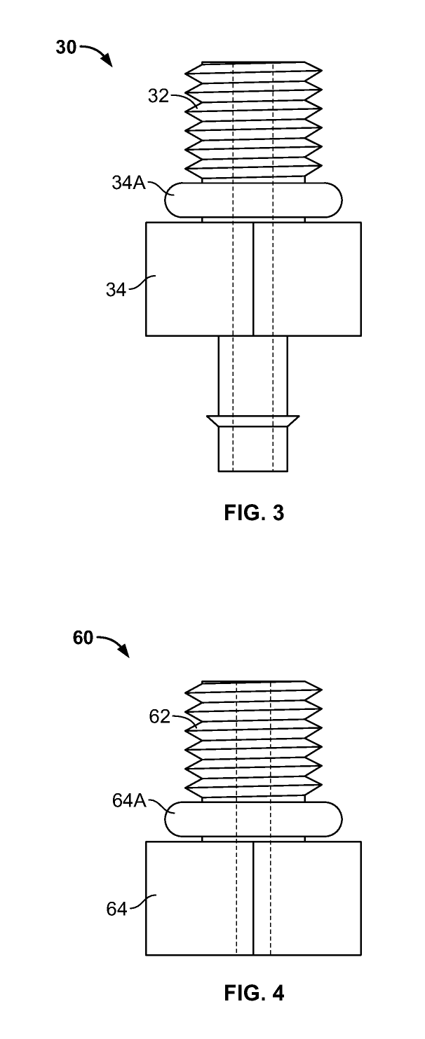

First end 12 of barrel 10 may couple with a front fitting 30, such as by an externally threaded section 32 of front fitting 30 threadedly engaging internally threaded first end 12 of barrel 10. Front fitting 30 may include externally threaded section 32, a body 34 (which may have a generally hexagonal cross section), and/or a nipple 36. Front fitting 30 may include an axial through passage 38, which may extend through nipple 36, body 34, and externally threaded section 32. As illustrated in FIG. 3, some example embodiments may include a seal, such as an o-ring 34A, which may provide a substantially sealed (e.g., substantially air tight) interface between front fitting 30 and barrel 10 when assembled. In some example embodiments, o-ring 34A may be disposed between externally threaded section 32 and body 34.

Returning to FIG. 1, a bias, such as helical coil compression spring 40, may be disposed in interior 16 of barrel 10, such as via second end 14 of barrel 10. When installed, a first end 42 of spring 40 may be arranged to press against set screw 20 and/or front fitting 30.

A valve disc, such as resilient (e.g., rubber) stopper 50, may be disposed in interior 16 of barrel 10, such as via second end 14 of barrel 10. A first end 52 of stopper 50 may be arranged to press against a second end 44 of spring 44. In some example embodiments, a second end 54 of stopper 50 may be generally conically tapered and/or may include a projection.

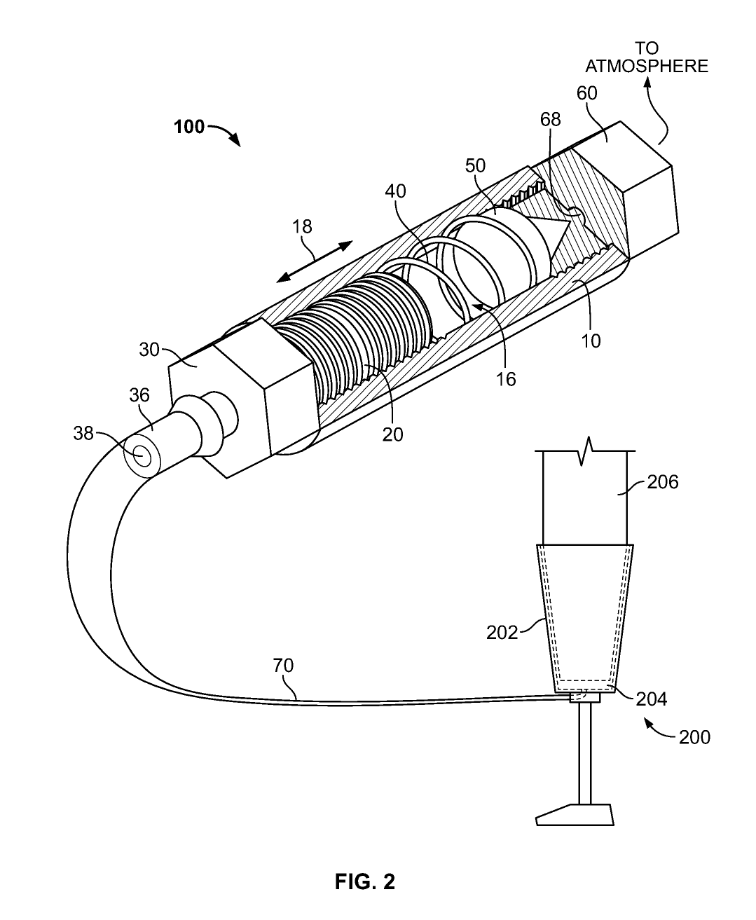

Second end 14 of barrel 10 may couple with a back fitting 60 (providing seal seat as discussed below), such as by an externally threaded section 62 of back fitting threadedly engaging internally threaded second end 14 of barrel 10. Back fitting 60 may include externally threaded section 62 and a body 64, which may have a generally hexagonal cross section. Back fitting 60 may include an axial through passage 68 (see FIG. 2), which may extend through body 64 and externally threaded section 32. Through passage 68 may open into a seat 66, which may be disposed within interior 16 of barrel 10 when assembled. Seat 66 may be shaped to releasably seal with second end 54 of stopper 50. For example, seat 66 may be inwardly generally conically tapered and/or may include a conical seating surface. Spring 40 may be arranged to bias stopper 50 against back fitting 60, such that the engagement between stopper 50 and back fitting 60 may be substantially air-tight. As illustrated in FIG. 4, some example embodiments may include a seal, such as an o-ring 64A, which may provide a substantially sealed (e.g., substantially air tight) interface between back fitting 60 and barrel 10 when assembled. In some example embodiments, o-ring 64A may be disposed between externally threaded section 62 and body 64.

FIG. 2 is a partial cutaway perspective view of an example bleeder valve assembly 100 according to at least some aspects of the present disclosure. When bleeder valve assembly 100 is assembled, set screw 20, spring 40, and/or stopper 50 may be disposed substantially entirely within interior 16 of barrel 10. Front fitting 30 and back fitting 60 may be disposed at first end 12 and second end 14 of barrel 10, respectively. Through passage 38 of front fitting 30 and through passage 22 of set screw 20 may be fluidicly connected such that interior 16 of barrel 10 may be fluidicly connected to nipple 36 of front fitting 30. Through passage 68 of back fitting 60 may be fluidicly connected to the ambient atmosphere.

In some example embodiments, a difference in pressure between ambient and nipple 36 (specifically through passage 38) may actuate bleeder valve assembly 100. Ambient pressure may act on stopper 50 via through passage 68 in back fitting 60. In particular, ambient pressure may oppose the force applied by spring 40 on stopper 50. In some example embodiments, the force applied by spring 40 on stopper 50 may tend to seat stopper 50 into a sealed engagement with back fitting 60 and/or the force of the ambient pressure on stopper 50 may tend to unseat stopper 50 from back fitting 60.

In some example embodiments, the force applied to stopper 50 by the ambient pressure may depend on the pressure difference between ambient pressure and a pressure in interior 16 of barrel 10, which is fluidicly connected to nipple 36. When the force applied to stopper 50 by the ambient pressure exceeds the force applied to stopper 50 by spring 40, stopper 50 may unseat from back fitting 60, thereby allowing airflow through bleeder valve assembly 100. When the force applied to stopper 50 by the ambient pressure is less than the force applied to stopper 50 by spring 40, stopper may seat against back fitting 60, thereby preventing airflow through bleeder valve assembly 100.

In some example embodiments, nipple 36 may be fluidicly coupled to an interior 204 of a socket 202 of a prosthetic limb 200, such as via a conduit 70. A vacuum may be applied to interior 204 of socket 202, which may aid in retaining socket 202 on a wearer's residual limb 206.

In some example embodiments, a bleeder valve assembly 100 may be arranged to prevent application of excessive vacuum to interior 204 of socket. For example, with nipple 36 fluidicly coupled to interior 204 of socket 202 by conduit 70, bleeder valve assembly 100 may be actuated based substantially upon a difference between ambient pressure and a pressure in interior 204 of socket 202. When this difference in pressure exceeds a predetermined set point, bleeder valve assembly may allow ambient air to enter interior 204 of socket 202.

In some example embodiments, a set point of a bleeder valve assembly may be adjusted and/or selected. For example, the force applied by spring 40 to stopper 50 may be adjusted by inwardly and/or outwardly threading set screw 20 in interior 16 of barrel in the directions generally indicated by arrow 18. In some example embodiments, spring 40 may be selected to provide a desired force on stopper 50. For example, a particular spring 40 having a desired spring constant may be selected from among a plurality of available springs having different spring constants. In some example embodiments, a spring 40 having a desired spring constant may be installed and the set point may be adjusted by varying the position of set screw 20.

As used herein, “set point” may refer to a pressure value or a pressure range at which bleeder valve assembly may actuate. For example, some embodiments according to the present disclosure may be provided in three configurations, each of which is associated with actuation at a different predetermined pressure range, such as 20-15 in Hg, 15-10 in Hg, and/or 10-5 in Hg. A prosthetist may determine the most appropriate pressure range for a particular patient and may select an appropriately configured bleeder valve assembly.

The present disclosure may be related to, and hereby incorporates by reference the following U.S. Patent documents: U.S. Provisional Patent Application No. 61/131,457, filed Jun. 9, 2008; U.S. Pat. No. 7,927,377, issued Apr. 19, 2011; U.S. patent application Ser. No. 12/897,807, filed Oct. 5, 2010; U.S. patent application Ser. No. 13/088,612, filed Apr. 18, 2011; and U.S. patent application Ser. No. 13/088,640, filed Apr. 18, 2011.

While example embodiments have been set forth above for the purpose of disclosure, modifications of the disclosed embodiments as well as other embodiments thereof may occur to those skilled in the art. Accordingly, it is to be understood that the disclosure is not limited to the above precise embodiments and that changes may be made without departing from the scope. Likewise, it is to be understood that it is not necessary to meet any or all of the stated advantages or objects disclosed herein to fall within the scope of the disclosure, since inherent and/or unforeseen advantages may exist even though they may not have been explicitly discussed herein.

What is claimed is:

1. A prosthetic limb socket assembly including a valve configured to automatically adjust vacuum pressure within the prosthetic limb socket based upon a difference in ambient air pressure and vacuum pressure within the prosthetic limb socket.

2. The prosthetic limb socket assembly, wherein the valve comprises a bleeder valve.

3. The prosthetic limb socket assembly of claim 2, wherein the bleeder valve includes:
a housing having a first end, a second end, and channel extending therethrough providing fluid communication between the ambient and the interior of the prosthetic limb socket;
a valve disc disposed within the channel and biased into a substantially sealed engagement with a valve seat, wherein a certain pressure difference between the ambient and the interior of the prosthetic limb socket is operative to overcome the bias and unseat the valve disc from the valve seat.

4. The prosthetic limb socket assembly of claim 3, wherein the bias is manually adjustable.

5. The prosthetic limb socket assembly of claim 3, wherein the bias is provided by a spring positioned within the channel between the valve disc and a spring seat.

6. The prosthetic limb socket assembly of claim 5, wherein the spring seat is threadedly received within the channel so that rotating of the spring seat moves the spring seat towards or away from the valve disc, depending upon direction of rotation of the spring seat, thereby respectively compressing or decompressing the spring.

7. A bleeder valve assembly for use with a prosthetic limb socket assembly, comprising a housing having a first end, a second end, and an interior channel, the housing being adapted to be mounted to a prosthetic limb socket assembly such that the interior channel provides fluid communication between the ambient and an interior of a prosthetic limb socket;
a spring seat positioned approximate the first end of the interior channel;
a valve seat positioned approximate the second end of the interior channel;
a valve disc positioned adjacent the valve seat; and
a spring positioned between the valve disc and the spring seat, biasing the valve disc into sealed contact with the valve seat, thereby sealing the interior channel;
wherein a certain pressure difference between the ambient and the interior of the prosthetic limb socket is operative to overcome the bias of the spring and unseat the valve disc from the valve seat.

8. The bleeder valve assembly of claim 7, wherein the spring seat is axially adjustable to allow for adjustment of the bias applied by the spring.

9. The bleeder valve assembly of claim 8, wherein the spring seat is threadedly mounted with respect to the interior channel.

10. The bleeder valve assembly of claim 7, wherein the housing is barrel shaped.

11. The bleeder valve assembly of claim 7, further comprising a first fitting disposed on the first end of the housing, the first fitting including a nipple and a seal between the first fitting and the housing.

12. The bleeder valve assembly of claim 11, further comprising a second fitting disposed on the second end of the housing and a seal disposed between the second fitting and the housing.

13. The bleeder valve assembly of claim 7, wherein the valve disc includes a resilient stopper.

14. The bleeder valve assembly of claim 13, wherein the valve disc includes a conical portion coaxial with the axis of the interior channel and tapering inwardly towards the first end.

15. A method for regulating vacuum pressure within a prosthetic limb socket assembly comprising automatically adjusting vacuum pressure within the prosthetic limb socket based upon a difference in ambient air pressure and vacuum pressure within the prosthetic limb socket.

16. The method of claim 15, wherein the adjusting step is performed by a valve assembly mounted to the prosthetic limb socket, the valve assembly providing fluid communication between the prosthetic limb socket interior and the ambient when in an unsealed orientation.

17. The method of claim 16, wherein the valve assembly is a bleeder valve assembly.

18. The method of claim 17, wherein the bleeder valve includes:
a housing having a first end, a second end, and channel extending therethrough providing fluid communication between the ambient and the prosthetic limb socket interior;
a valve disc disposed within the channel and biased into a substantially sealed engagement with a valve seat, wherein a certain pressure difference between the ambient and the interior of the prosthetic limb socket is operative to overcome the bias and unseat the valve disc from the valve seat.

19. The prosthetic limb socket assembly of claim 18, wherein the bias is manually adjustable.

20. The prosthetic limb socket assembly of claim 19, further comprising manually adjusting the bias.
